(19) United States
(12) Patent Application Publication
(10) Pub. No.: US 2018/0049715 A1
(43) Pub. Date: Feb. 22, 2018

(54) Automated Identification and Selection of a Region of Interest in Imaging

(71) Applicant: Straxcorp Pty Ltd, Melbourne (AU)

(72) Inventors: Roger ZEBAZE, Coburg (AU); Yu PENG, Box Hill (AU)

(21) Appl. No.: 15/560,919

(22) PCT Filed: Mar. 23, 2016
(86) PCT No.: PCT/AU2016/050210

§ 371 Date: Sep. 22, 2017

(30) Foreign Application Priority Data
Mar. 23, 2015 (AU) 2015901027
Jul. 9, 2015 (AU) 2015902714

(51) Int. Cl. A61B 6/00; A61B 6/03

(52) U.S. Cl. CPC A61B 6/582; A61B 6/505; A61B 6/032; A61B 6/469

(57) ABSTRACT

A method and apparatus for calibrating an image of a specimen or subject obtained with an imaging modality or selecting a region of interest in the image. The apparatus comprises: a graduation support adapted for mounting on the specimen or subject; and one or more calibration graduations supported by the graduation support; the one or more calibration graduations are imagable with the imaging modality, and are distinguishable in the image from the graduation support and from the specimen or subject, and at least one of the calibration graduations have at least one characteristic of known value.

### RELATED APPLICATIONS

This application is based on and claims the benefit of the filing and priority dates of Australian patent applications Nos. 2015901027 filed 23 Mar. 2015 and 2015902714 filed 9 Jul. 2015, the content of which as filed is incorporated herein by reference in its entirety.

### FIELD OF THE INVENTION

The present invention relates to a method and apparatus for automated identification or selection of a region of interest (ROI) of an image of a sample to be analyzed, such as to characterize the sample, identify the sample, or otherwise.

### BACKGROUND OF THE INVENTION

Among the many challenges in the analysis of an image of a sample, such as bone, is the selection of a suitable or appropriate region of interest (ROI) for the analysis.

For example, in the context of imaging a human forearm for quantification of its structure or density, such as for diagnostic purposes or the monitoring of treatment, it is important that in two individuals a common ROI relative to some suitable reference point (e.g. a joint line) be identified. That is, the ROI should be similar across individuals, and hence cover the same or similar macro- and micro-anatomy. If this criterion is not met, structure and density may appear to vary owing to the difference in the position of the ROI relative to the reference point rather than owing to actual differences (such as in density or architecture) between the two individuals. This can taint the results of the analysis and hence any diagnostic conclusions based on those results.

Some manufacturers of medical scanners instruct the user to select a ROI at a fixed distance from a suitable reference point or location, such as a joint. FIG. 1 is an X-ray image 10 of the distal end of a human radial bone, in which the location of the wrist joint 12 has been used as a reference 14. Image 10 is a so-called ‘scout view’, being an X-ray image taken with a CT scanner. Image 10 is shown in negative, in which bone is dark, for greater clarity. A region of interest 16 has then been established, with a predefined width, at a predefined distance 18 from reference 14. The ROI 16 is thus selected according to the position of reference 14. This approach is, however, subject to error and may lead to the selection of ROIs with innate differences in macro- and micro-anatomy. The results of the subsequent analysis may then be tainted by error with the consequences described above, that is, owing to variations in bone architecture varies along the length of the bone. A bone is made of thin cortex close to a joint, but towards a bone's centre there are a large number of trabeculae. Moving proximally, the cortex becomes thick and trabeculae disappear so that, at its midshaft, the bone is almost exclusively made of cortical bone. This transition is shown in FIG. 2, comprising CT images of the radial bone 22 shown in FIG. 1 (also shown in negative for greater clarity). In the lower register is a CT image 20 showing (at distal end 24) the wrist joint end of bone 22 and (towards proximal end 26) the midshaft portion of bone 22. Upper left register 28 of FIG. 2 is a cross-sectional CT image of bone 22 through line A-A, while upper right register 30 of FIG. 2 is a cross-sectional CT image of bone 22 through line B-B. The bone density near its central portion (cf. upper left register 28) is clearly far greater than that towards the wrist joint (cf. upper right register 30). Indeed, the transition from thin cortices with large numbers of trabeculae adjacent to the joint, to thick cortices and very few trabeculae near the centre of the bone, occurs in an exponential manner. As a result, and because subjects (such as people) differ significantly in size, any ROI selected as a fixed distance from a joint line is likely to encompass different macro- and micro-anatomy from subject to subject. This problem is compounded by the fact that the selection of the ROI in this approach is done manually, so prone to reproducibility error.

Mindful of this problem, others have proposed the selection of the ROI as a percent of total bone length relative to a reference point (typically a joint's midline). This approach is typically better than the fixed distance ROI approach described above, but it requires the technician or health professional to (i) measure the length of the bone accurately, (ii) calculate the correct distance from the reference point and (iii) use the calculated distance to determine the correct ROI. This approach is cumbersome, time consuming, and subject to reproducibility error being—again—operator dependent.

### SUMMARY OF THE INVENTION

According to a first broad aspect of the invention, there is provided an apparatus for calibrating an image of a specimen or subject obtained with an imaging modality (including X-ray and CT imaging) or selecting a region of interest in the image, the apparatus comprising:
a graduation support adapted for mounting on the specimen or subject; and
one or more calibration graduations supported by the graduation support;
wherein the one or more calibration graduations are imagable with the imaging modality, and are distinguishable in the image from the graduation support and from the specimen or subject (and, if there are plural graduations, optionally distinguishable for each other); and
   wherein at least one of the calibration graduations (and, in some embodiments, more than one or all of the calibration graduations) have at least one characteristic of known value.

In some embodiments, known values of one or more characteristics of the specimen or subject are required for region of interest selection, but in other embodiments the one or more characteristics of the specimen or subject need not have known values for region of interest selection.

The graduation support may be a member (which may be elongate), or a wearable item.

In some embodiments, selecting the region of interest may require prior determination of one or more physical characteristics of the specimen or subject, such as a length or width of the specimen or subject.

In some embodiments, selecting the region of interest may not require prior determination of one or more physical characteristics of the specimen.

When prior determination of one or physical characteristics of the specimen or subject is required, the graduation support may be an elongate member that is extensible or compressible between a relaxed form and an extended or compressed form.

In one embodiment, there is provided an apparatus for calibration of an image of a specimen or subject, the apparatus comprising:
a graduation support comprising an elongate member extensible or compressible between a relaxed form and an extended or compressed form; and
the one or more calibration graduations are provided along the elongate member;
wherein the calibration graduations are imagable with the imaging modality, and are distinguishable in the image from the elongate member and from the specimen or subject (and optionally distinguishable for each other); and
wherein at least some of the calibration graduations (and, in some embodiments, all of the calibration graduations) have at least one characteristic of known value in the relaxed form and of determinable value from the image.

It should be understood that the term “compressed” need not imply that a compressive force arises upon compression; for example, the elongate member may comprise a telescopic member or arrangement that does not significantly resist compression (other than owing to friction).

It will be understood that the known value and the determinable value when determined from the image may be different, such as due to elongation of the apparatus in use.

The graduation support may be resilient, such as when calibration is required for selecting the region of interest.

In one embodiment, the graduation support (which may be in the form of an elongate member) is compressible between a relaxed form and a compressed form, and comprises one or more extensible and/or retractable elongate structures.

The graduation support may comprise an elastic material, one or more springs (such as woven into or mounted on a fabric) or a telescopic mechanism. The springs may be extensible and/or compressible.

In a particular embodiment, the graduation support is extensible between the relaxed form and an extended form and compressible between the relaxed form and a compressed form (such as by comprising a spring whose relaxed form corresponds to the relaxed form of the graduation support).

Hence, in its relaxed form, the graduation support may have a length between the extremes of the expected lengths of the specimen or subject, and—in use—be extended for use with longer specimens or compressed for use with shorter specimens.

In one embodiment, the calibration support has only one calibration graduation. In another embodiment, the calibration support has a plurality of calibration graduations.

In one embodiment, an identity of at least one of the calibration graduations is determinable from the image. This may be done, for example, on the basis of the at least one characteristic of known value.

In one embodiment, the at least one characteristic is the known spatial position (which may be expressed in relative or percentage terms) of the one or more graduations.

In one embodiment, the at least one characteristic is a separation of each of the calibration graduations from one or more immediately neighbouring calibrations. In an embodiment, the at least one characteristic is the number of graduations at predetermined (i.e., known) spatial positions.

In another embodiment, the at least one characteristic is a length of at least some of the calibration graduations. In some embodiments, each of the calibration graduations is unique.

In some embodiments, the apparatus is generally tubular (at least in use) and the calibration graduations are arranged circumferentially. In these embodiments, the calibration graduations may be generally arcuate (though this may be so, or clearly apparent, only in use). In some embodiments, a predetermined of calibration graduation are arranged circumferentially

In certain embodiments, the calibration graduations are arranged longitudinally (such as at equal intervals around or mounted on a generally tubular portion of the apparatus, or at equal intervals on part or whole perimeter of a cross section of the apparatus).

In an embodiment, the apparatus is generally tubular (at least in use), some of the calibration graduations are arranged circumferentially and some of the calibration graduations are arranged longitudinally.

In one embodiment, the calibration graduations have different densities from each other, and/or a different density or densities from a density of the graduation support and a density of the specimen or subject in order to be distinguishable in the image from the graduation support and from the specimen or subject.

In one embodiment, the one or more calibration graduations are located at respective predetermined spatial positions so as to be distinguishable in the image from the graduation support and from the specimen or subject (and thereby allow ease of segmentation).

In one embodiment, the one or more calibration graduations are located at respective predetermined spatial positions so that the spatial positions can be used as respective referential points.

In a certain embodiment, the apparatus comprises a fastener for fastening the graduation support to a specimen or subject in the extended or compressed form. The fastener may be integral with the graduation support. The fastener may comprise a first tie or cord lock located or locatable at a first end of the graduation support, and a second tie or cord lock located or locatable at a second end of the graduation support. The first and second ties may be in the form of bands (such as of an elastic or inelastic material).

The first and/or second ties may be fastened using any suitable mechanism, such as one or more cord locks, Velcro (trade mark) taps or Velcro straps.

According to a second broad aspect of the invention, there is provided a method for calibrating an image of a specimen or subject obtained with an imaging modality (including X-ray and CT imaging) or selecting a region of interest in the image, the method comprising:
locating an apparatus as described above on a subject or specimen;
imaging at least a portion of the subject or specimen containing one or more of the calibration graduations;
determining from the image the at least one characteristic of one or more of the calibration graduations; and
selecting the region of interest.

The method may include comparing the at least one characteristic of the one or more of the calibration graduations in the image with known values of the respective one or more calibration graduations in the image and determining a calibration therefrom.

In one embodiment, there is provided a method for calibration of an image of a specimen or subject, the method comprising:
locating an apparatus for calibrating an image of a specimen or subject obtained with an imaging modality or selecting a region of interest in the image as described above;
imaging at least a portion of the subject or specimen containing at least one of the calibration graduations;
determining from the image the at least one characteristic of one or more of the calibration graduations; and
comparing the at least one characteristic of the one or more of the calibration graduations in the image with known values of the respective one or more calibration graduations in the image and determining a calibration therefrom.

The graduation support may comprise an elongate member. Locating the apparatus on the subject or specimen, or calibrating the image, may include extending or compressing the elongate member.

Comparing the at least one characteristic of the one or more of the calibration graduations in the image with known values of the respective one or more calibration graduations in the image may involve determining, for example, a ratio of relaxed and extended or compressed separations of one or more pairs of calibration graduations, or determining a ratio of relaxed and extended or compressed lengths or widths of one or more calibration graduations, or counting the number of graduations arranged circumferentially, longitudinally, or any other direction.

In one embodiment, an identity of at least one of the calibration graduations is determinable from the image. This may be done, for example, on the basis of the at least one characteristic of known value (e.g. length, width or density, or length, width or density relative to another of the calibration graduations).

In one embodiment, the at least one characteristic is a separation of each of the calibration graduations from one or more immediately neighbouring calibrations.

In another embodiment, the at least one characteristic is a length of at least some of the calibration graduations. In some embodiments, each of the calibration graduations is unique. In still another embodiment, the at least one characteristic is the number of calibration graduations in a given direction (e.g., circumferentially).

In another embodiment, the graduation is unique and identifiable by at least one of its characteristics (e.g., density and/or spatial position).

In one embodiment, the method includes automatically selecting or identifying a region of interest for imaging based on the calibration and a predefined or desired imaging location.

The calibration graduations may have at least two different known densities, and the method may include calibrating a specimen or subject density based on the at least two different known densities of the calibration graduations.

According to a third broad aspect of the invention, there is provided a system for calibrating an image of a specimen or subject obtained with an imaging modality (including X-ray and CT imaging) or selecting a region of interest in the image, the system comprising:
an input for receiving an image of the specimen or subject and of the calibration apparatus described above;
a calibration graduation locater for locating one or more calibration graduations in the image (such as with a thresholding method or other methods of image segmentation);
a graduation segmenter for segmenting the calibration graduations in the image;
a graduation identifier for identifying at least one characteristic of the calibration graduations in the image; and
a calibrator for preparing a calibration of the image based on the at least one characteristic of the calibration graduations in the image (and optionally also on known values of the respective one or more calibration graduations in the image).

The calibration may be output by the calibration system, or used by the calibration system in subsequent image analysis.

The calibration graduations may have at least two different known densities, and the calibrator may be configured to calibrate a specimen or subject density based on the at least two different known densities of the calibration graduations.

According to a fourth broad aspect of the invention, there is provided an image analysis system, comprising a system according to the third aspect.

According to a fifth broad aspect of the invention, there is provided an image analysis system, comprising a graduation support with one or more graduations according to the first aspect.

According to a sixth broad aspect of the invention, there is provided an imaging system, comprising a system according to the third aspect.

In one embodiment, the imaging system is configured to automatically select or identify a region of interest for imaging based on the calibration and a predefined or desired imaging location.

In another embodiment, a user manually selects the region of interest based on the information provided by the graduation support and the characteristics of the graduation visible in the imaged portion of the subject.

According to a seventh broad aspect of the invention, there is provided a computer software product configured to control one or more processors to implement the calibration method described above. This aspect also provides a computing device provided with the computer software product.

It will be understood that the image will commonly be in digital form, so the term “image” is used to refer to the image whether in digital form or otherwise, and hence whether viewable at any particular time or not. It should also be borne in mind that an image generated by a CT scanner typically comprises a plurality of CT slices, each of which may be displayed and viewed individually if desired.

It should be noted that any of the various individual features of each of the above aspects of the invention, and any of the various individual features of the embodiments described herein including in the claims, can be combined as suitable and desired.

### BRIEF DESCRIPTION OF THE DRAWING

In order that the invention may be more clearly ascertained, embodiments will now be described, by way of example, with reference to the accompanying drawing, in which:

FIG. 1 is an X-ray image according to the background art of a portion of a human radius;

FIG. 2 comprises CT images according to the background art of portions of a human radius;

FIG. 3A is a schematic view of an image ruler apparatus according to an embodiment of the present invention, in relaxed form;

FIG. 3B is a schematic view of the image ruler apparatus of FIG. 3A, in extended (in-use) form;

FIG. 4 is a schematic view of an imaging system including image analyzer, according to an embodiment of the present invention;

FIG. 5 is a schematic view of the image analyzer of the system of FIG. 4;

FIG. 6 is a schematic view of the memory of the processing controller of the image analyzer of the system of FIG. 4;

FIG. 7 is another, more detailed schematic view of the image analyzer of the system of FIG. 4;

FIG. 8 is a flow diagram illustrating a calibration process according to an embodiment of the present invention;

FIG. 9A is a schematic view of a wearable image ruler apparatus according to another embodiment of the present invention;

FIG. 9B is a schematic view of a 3D referential coordinate system showing how the position of the region of interest is identified with the apparatus of FIG. 9A;

FIG. 10A is a schematic view of the image ruler apparatus according to an embodiment of the present invention in a relaxed form; and

FIG. 10B is a schematic view of the image ruler apparatus of FIG. 10A in an extended, in use form.

### DETAILED DESCRIPTION OF THE INVENTION

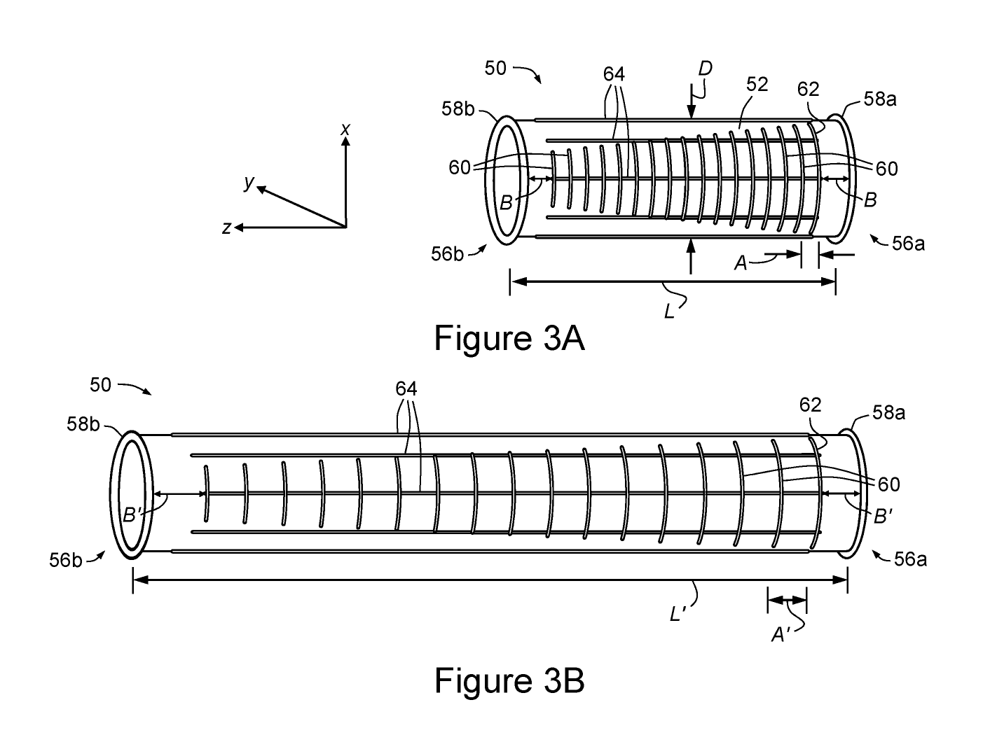

FIG. 3A is a schematic view of an image ruler apparatus 50 according to an embodiment of the present invention. Image ruler apparatus 50 is approximately anatomically shaped (in this example for a human forearm), with generally tubular main section 52 of a bandage-like (and hence generally woven) material, such that it is resilient in both the in-plane (x-y) direction and in the longitudinal (z) direction. In this embodiment, image ruler apparatus 50 has two forms: a relaxed or unextended form as shown in FIG. 3A, and an extended form as shown in FIG. 3B, as is typically the case.

Apparatus 50 is designed to be worn on and around the forearm, and the aforementioned in-plane resilience allows apparatus 50 to fit forearms of different sizes. Apparatus 50 has a relaxed length L selected not to exceed the length of the relevant bone, limb, etc (in this example, the forearm) and typically 50% of that length, so that it can be extended along the forearm without exceeding the length of the forearm. The average length of an adult forearm is about 23 cm, so—in this embodiment—relaxed length L is 10 cm. In this embodiment, apparatus 50 is designed to stretch resiliently to at least twice its relaxed length and desirably to at least 2.5 times its relaxed length, to accommodate the vast majority of human subjects. The relaxed diameter D of main section 52 is selected so that the circumference of main section 52 is able to accommodate the majority of human forearms with only a little stretching. If D is too low, it would be necessary for apparatus 50 to be highly stretched circumferentially to accommodate the subject's forearm. Depending on the material of main section 52, this may result in a large force between apparatus 50 and the forearm and hence significant friction; this may produce a tendency for apparatus 50 not to extend uniformly when extended along the subject's forearm, and create a risk of impeded or occluded blood circulation and discomfort. Apparatus 50 should also not be too loose, as circumferential graduations 62 (described below) may then not—in use—reflect the anatomy of the forearm. For example, circumferential graduations 62 may be linear after extension rather than curved around the curved perimeter of the forearm. A suitable perimeter of apparatus 50 in its relaxed form, for any particular size forearm or range of size, will be readily established, at a minimum by straightforward trial.

Apparatus 50 includes, at its distal (or wrist) end 56a and proximal end 56b, fasteners in the form of elastic bands 58a, 58b respectively for fastening apparatus 50 to the forearm in use. In particular, elastic band 58a is adapted to hold apparatus 50 in place at the wrist joint, such as to the radius styloid process, though any other region indicative of the wrist joint will also generally be acceptable if used consistently. Any other suitable fasteners may be employed, such as straps or ties.

Once apparatus 50 has been placed around the forearm and fastened securely at the wrist joint, it is extended and hence deployed proximally towards the elbow joint, then fastened with elastic band 58b firmly at the elbow joint, such as above the olecranon process. As with attachment at the wrist joint, other region indicative of the elbow joint will also be acceptable, if used consistently.

Apparatus 50 includes two sets of graduations for allowing the determination of its degree of extension. The first or circumferential graduations 60 are in the form of members—such as fibers—embedded in the main section 52 of apparatus 50 and extending circumferentially essentially parallel to one another. Main section 52 thus acts as a graduation support. Graduations 60 are shown as arcuate, but they may be flexible and hence only arcuate once in use on a subject's forearm.

Circumferential graduations 60 are of a material with a density sensibly different from that of main section 52 and of the soft biological tissue of the forearm, but lower than that of bone matrix. This is so that circumferential graduations 60 are distinguishable in CT images without affecting the imaging of bone (such as through beam hardening or Compton scattering). In this embodiment, circumferential graduations 60 are in the form of a wearable material, such as cotton fibers (which should be sufficiently inelastic if stitched firmly to main section 52), that has a density of ˜1.55 g/cm³ (soft tissue having a density of ˜1.08 g/cm³). Circumferential graduations 60 may alternatively be made of wool, plastic or cotton tube-like cylinders filled with a hydroxyapatite of density superior greater than that of soft tissue but less than that fully mineralized bone matrix (typically ˜400 mgHA/cm³) or filled with calcium or phosphate powder.

This allows graduations 60 to be readily visible without interfering with image acquisition. It should be noted that the concentration in such examples need not be known exactly, provided circumferential graduations 60 can be identified in an image rather than accurately determined from that image. Generally, therefore materials with a density that is not so great as to cause distortion of images, and not so low as to be difficult to discern in the images, are desirable.

As a result, another suitable material for graduations 60 may be limestone or other stones that are principally of calcium carbonate (CaCO₃) and thus similar to calcium hydroxyapatite, the main constituent of bone. In bone imaging applications, main section 52 may be, for example, of a plastics material (e.g. nylon) or any other non-metallic material that is different from the graduations.

The longitudinal spacing A between adjacent graduations is selected such that, when apparatus 50 is in its most extended in-use form, circumferential graduations 60 will be separated by no more than will ensure that at least two of circumferential graduations 60 appear in the typical field of view of an overview or scouting image employed in the intended type of imaging. Consequently, in this embodiment, which is intended for use in obtaining CT images, A is 2 mm.

The first (that is, most distal) circumferential graduation 62 of circumferential graduations 60 is located at a distance B from distal end 56a (and hence, in use, the wrist joint). The distance B is somewhat arbitrary, but is selected such that in use there are graduations 60 over as great a length of the forearm as reasonably possible, especially over portions of the forearm likely to require imaging. The distance between the proximal-most of graduations 60 and proximal end 56b is, in this embodiment, also B. In this embodiment, B is 2 mm.

Hence, in the present embodiment, A and B are both 2 mm; the relaxed length L of apparatus 50 is 10 cm, so apparatus 50 has 49 circumferential graduations 60. It will be noted that FIGS. 3A and 3B are not to scale; for example, only 17 exemplary circumferential graduations 60 are depicted.

Each of circumferential graduations 60 differs from at least its immediate neighbour or neighbours, to facilitate identification of circumferential graduations 60 when captured in an image. The degree to which circumferential graduations 60 should vary to achieve this aim depends on how precisely the imaging apparatus with which apparatus 50 is to be employed can identify image location without apparatus 50. If this is possible to a reasonably high level of precision, it may be sufficient that each of circumferential graduations 60 differ from only its immediate neighbour or neighbours. This may be done by having circumferential graduations 60 alternate in width (i.e. in the z direction) or in length (i.e. circumferentially) with their immediate neighbours. For example, in one embodiment, circumferential graduations 60 all have a length of 1 cm, but each of the first, third, fifth, etc. circumferential graduations 60 has a width of 0.5 mm, and each of the second, fourth, sixth, etc. circumferential graduations 60 has a width of 0.3 mm.

In another embodiment, all of circumferential graduations 60 have a width of 0.5 mm, but the first, third, fifth, etc. circumferential graduations 60 have a length of 1 cm, while the second, fourth, sixth, etc. circumferential graduations 60 have a length of 0.5 cm. In other embodiments, circumferential graduations 60 may need to be distinguishable from at least two (or more) neighbouring graduations on each side.

In some embodiments, the distinguishability of circumferential graduations 60 is provided by making circumferential graduations 60 of material that is distinguishability in the intended imaging modality.

In some embodiments, however, circumferential graduations 60 are all distinguishable from one another, so that the identity of any one of circumferential graduations 60 can be determined from its dimensions. As shown schematically in FIG. 3A, this is done in the present embodiment using the circumferential length of circumferential graduations 60, as apparent in a cross-sectional view of the bone (cf. upper left register 28 of FIG. 2). Referring to FIG. 3A, the circumferential length of circumferential graduations 60 starts (at first circumferential graduation 62) at—for example—5.8 cm and diminishes with each successive circumferential graduation 60 by—for example—0.1 cm, such that the proximal-most of circumferential graduations 60 has a length of 1 cm. In some embodiments circumferential graduations 60 have a fixed width but vary (such as by monotonically increasing or decreasing) in length as a function of their position relative to the reference point, such as according to a predefined mathematical function. For example, circumferential graduations 60 may start with a length of L₁ for first circumferential graduation 62, with the length Lₙ of circumferential graduation n being Lₙ=L₁+k×n×L₁, where k is a coefficient (e.g., 10%) and n is the graduation's rank, n=1, 2, 3, . . . .

In another embodiment, circumferential graduations 60 are all made distinguishable from one another using their width in the z direction. First circumferential graduation 62, in such an embodiment, has a width of 0.5 mm, and the width of increases for each successive circumferential graduation 60 by 0.3 mm, such that the proximal-most of circumferential graduations 60 would have a width of 1.49 cm. In some embodiments, circumferential graduations 60 may have a fixed length but increase (or decrease) in width as a function of their position relative to the reference point, such as according to a predefined mathematical function. For example, circumferential graduations 60 may start with a width of W₁ for first graduation 62, the width Wₙ of graduation n being Wₙ=W₁+k×n×W₁, where k is a coefficient (e.g., 10%) and n is again the circumferential graduation's rank.

In still another embodiment, this two last approaches are combined, such that circumferential graduations 60 increase or diminish in length and increase or diminish in width over the length of apparatus 50. Indeed, distinguishability of circumferential graduations 60 may be providing as many of these (and other) approaches as desired or required.

In use circumferential graduations 60 are generally inextensible as apparatus 60 stretches, in at least the direction relied upon to distinguish each circumferential graduations 60 from one or more others, so that their true width and/or length is not distorted by such stretching. Thus, if width in the z direction is used to distinguish circumferential graduations 60, circumferential graduations 60 are generally inextensible in the z direction, while in embodiments in which circumferential length is used to distinguish circumferential graduations 60, circumferential graduations 60 are generally inextensible circumferentially. In embodiments in which both dimensions are employed for this purpose, circumferential graduations 60 are generally inextensible in both length and width.

Such inextensibility can be accomplished in a number of ways, including making circumferential graduations 60 of a generally inextensible material or by mounting graduations 60 to main section 62 in a manner that does not transfer extensive force (at least in the relevant direction or directions, i.e. the z direction, circumferentially, or both) to graduations 60 when apparatus 60 is stretched. In the latter example, this may be effected by weaving circumferential graduations 60 into main section 62; main section 52 is of a woven material, so circumferential graduations 60 will be able—to a sufficient extent—to slide within the fabric of main section 52 as it expands in circumference in use. Each of circumferential graduations 60 may be additionally fastened to main section 52 near its centre, such as by stitching, so that its absolute position within apparatus 50 is even more securely maintained.

Apparatus 50 also includes a set of elongate longitudinal graduations 64, each extending in the z direction along main section 52 for a distance comparable to that covered by circumferential graduations 60.

Longitudinal graduations 64 are provided for facilitating the determination of position in 2D images, typically—in this embodiment—in the form of X-ray images. Longitudinal graduations 64 are arranged at even intervals near the surface of apparatus 50, such that they extend somewhat from the surface of main section 52, and can be resolved by the intended imaging modality. In this embodiment, as X-ray images have an approximate resolution of 500 μm, this interval should be approximately 5 mm or somewhat greater in use. Again, it will be noted that FIGS. 3A and 3B are not to scale; only eight exemplary longitudinal graduations 64 are suggested, but in this embodiment the actual number of longitudinal graduations 64 is 19—though it could be somewhat more according to the size of apparatus 50.

Longitudinal graduations 64 are of a material that is also distinguishable from the subject's tissues in the desired (typically X-ray) images, so may be of the same material as circumferential graduations 60. However, longitudinal graduations 64 are resilient in the z direction so that they extend with apparatus 50 when apparatus 50 is extended.

Although both circumferential graduations 60 and longitudinal graduations 64 are depicted as elongate members, but other configurations are possible. For example, in either case a sequence of discrete beads could be employed, of known separation in the relaxed state of apparatus 50. In addition, neither set of graduations 60, 64 need be of a material with the same density in all graduations 60, 64. Different densities, for example, may be used to distinguish circumferential graduations 60 from one another and, likewise, to distinguish longitudinal graduations 64 from one another.

FIG. 3B is a schematic view of apparatus 50 according to this embodiment in use. In use, apparatus 50 is worn by a subject on his or her forearm, fastened at the wrist joint, then stretched to the elbow joint and fastened. If the length of the forearm of the subject and hence extended length of apparatus 50 is L″, the spacing between each pair of adjacent circumferential graduations 60 (in relaxed form, A) increases by L″/L to A″=A×L′/L. The extent of longitudinal graduations 64 also increases by L″/L.

To obtain A″, CT data is collected and processed with an image analyzer (described below). During the image processing, the nth circumferential graduation (at distance G(n) from distal end 56a of apparatus 50 in its relaxed form) that appears in the ROI where the image has being collected is also identified by the image analyzer. This is done by segmenting circumferential graduation n from the image and graduating apparatus 50 (according to the width or length of the circumferential graduation n, or the density, as discussed above) to identify circumferential graduation n.

After identifying circumferential graduation n in the image, the ROI selector (described below) identifies the position P(n) of circumferential graduation n from distal end 56a (and hence relative to the wrist joint), where P(n)=G(n)×A′/A. From the referential point P(n), the position of any other point within the image can then be automatically determined by the ROI selector in absolute terms or relative to the length of the subject.

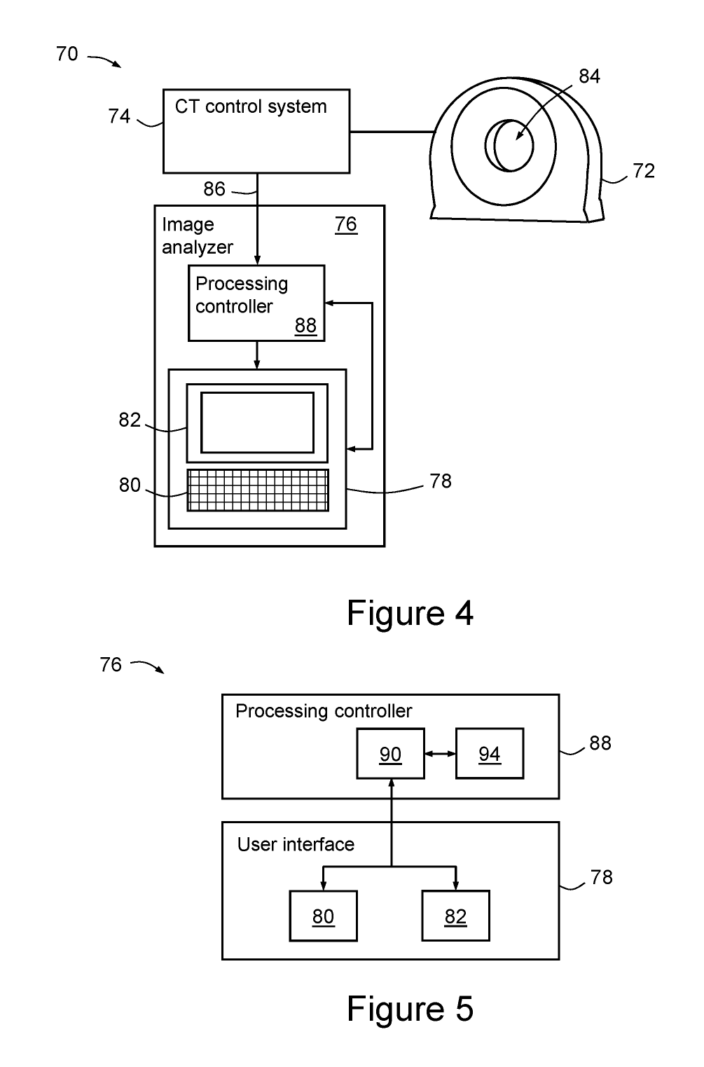

FIG. 4 is a schematic view of an imaging system 70 according to an embodiment of the present invention, and which includes the aforementioned image analyzer. Imaging system 70 comprises a CT scanner 72, CT control system 74 and an image processor or analyser 76, and operates as does the system for detecting fracture-vulnerable bone 10 of International Patent Application Publication No. WO 2011/029153(which is incorporated herein by reference). Briefly, however, image analyser 76 of system 70 includes a user interface 78 comprising an input device and an output device. The input device is in the form of a keyboard and mouse 80 for controlling image analyzer 76, and the output device is in the form of a display 82 for displaying images from the CT scanner 72 before and after processing by image analyzer 76, or alternatively for displaying the results as text to indicate to the user when the image has been collected or to suggest where a new image should be collected so that it contains a ROI.

The user interface may include a touch screen display, which serves both as an input device and as an output device.

CT scanner 72 is configured to perform CT scans of a sample located within central scanning volume 84 of CT scanner 72 and to output digitized scan data to CT control system 74; CT control system 74 is configured to generate image data from the data received from CT scanner 72, and to output these image data to image analyzer 76.

CT scanner 72 can operate in both a conventional X-ray imaging mode and in a CT imaging mode, so—in this embodiment—the image data comprises X-ray images and CT images, including image slices or strips through the sample. In other embodiments, other sections may be used (such as coronal, transverse or oblique sections).

System 70 may operate in online and off-line modes. In the online mode, image processor 76 receives data directly from CT control system 74 (during or soon after scanning of the sample). Such an arrangement may be used in a clinical setting. In this online mode, the data is transmitted to image analyzer 76 via a data link (connected to respective USBs of the

CT control system 74 and image analyzer 76) or a communications network (to which CT control system 74 and image analyzer 76 are both connected, such as in the form of the internet); this link or network is shown schematically in FIG. 4 at 86.

In the off-line mode, image analyzer 76 receives data collected earlier by CT scanner 72 and CT control system 74; the data may be transmitted to image analyzer 76 via communications link or network 86, or by any other suitable means (including on portable computer readable medium, such as CD-ROM, flash card or the like).

Image analyzer 76 includes a processing controller 88 that is in data communication with input 80 and output 82 and configured to process image processing instructions in accordance with a processing procedure (discussed below) and to output processing outcomes (which may comprise images and/or detection results) to display 82.

Referring to FIG. 5, processing controller 88 comprises a digital processor 90 that processes the processing instructions in accordance with the processing procedure and—and described above—outputs processing outcomes to display 82. Keyboard 80 and display 82 together constitute user interface 78. Typically, the processing instructions are stored as program code in a memory 94 of processing controller 88 but can also be hardwired. Herein the term “processor” is used to refer generically to any device that can process processing instructions in accordance with the processing procedure and may comprise: a microprocessor, microcontroller, programmable logic device or other computational device, a general purpose computer (e.g. a PC) or a server.

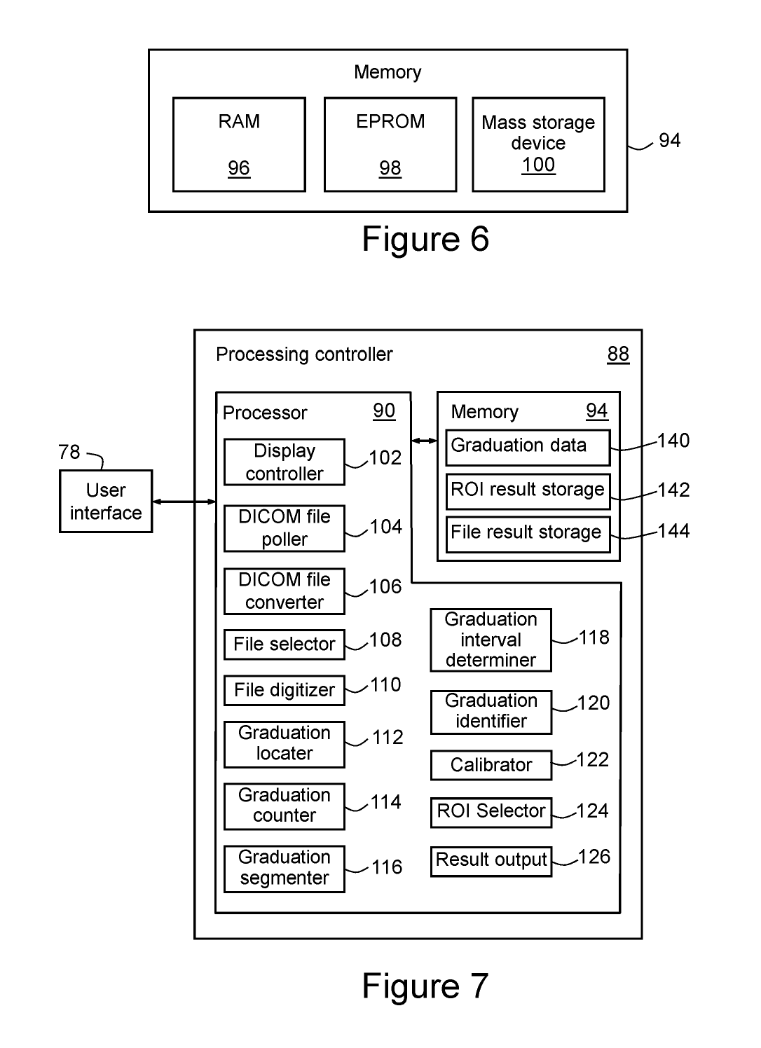

FIG. 6 shows a block diagram of the main components of memory 94. Memory 94 includes RAM 96, EPROM 98 and a mass storage device 100. RAM 96 typically temporarily holds program files for execution by the processor 90 and related data. EPROM 98 may be a boot ROM device and/or may contain some system or processing related code. Mass storage device 100 is typically used to store processing programs.

FIG. 7 is another schematic view of user interface 78 and processing controller 88 of system 70 of FIG. 4, with more detail shown in processing controller 88. Specifically, processor 90 of processing controller 88 includes a display controller 102 for controlling display 82, a file poller in the form of DICOM file poller 104 for polling DICOM (‘Digital Imaging and Communications in Medicine’) files or files in any other format, as desired, from a staging area (typically CT control system 74), a DICOM file converter 106 for converting DICOM files into images for display, a file selector 108 controllable to select the files to be processed by image analyzer 76, and a file digitizer 110 for digitizing the selected files.

Processor 90 also includes a graduation locater 112 for locating the graduations present in the image, a graduation counter 114 for counting calibration graduations in the image, a graduation segmenter 116 for segmenting calibration graduations in the image, a graduation interval determiner 118 for determining the interval between two (generally consecutive) calibration graduations, a graduation identifier 120 for identifying calibration graduations identified in the image, a calibrator 122 for calibrating the image, a ROI selector 124 (controllable to select an initial ROI to be analysed for the calibration and configured, as described below, to automatically select a ROI based on the calibration and a predefined point or region of interest), and a result output 126 for outputting results and messages.

The processing instructions that embody the processing procedure are in the form of program code stored in memory 94, and are adapted to control processing controller 86 to perform image processing. Briefly, image analyzer 76 receives (in either online or off-line mode) a DICOM file collected during a scan—whether in the form of an X-ray image or a CT scan—of a subject's forearm while that subject is wearing image ruler apparatus 50. Image analyzer 76 does this by polling CT control system 74 for such images, and image analyzer 76 stores the received file in memory 94. The received file, in this embodiment, is a DICOM file but in other embodiments may be in a different image format, such as JPEG or TIFF. Image analyzer 76 converts the file into an image and, if the user controls apparatus 70 to display the image, outputs the image to display 82 so that the user can view the image to be processed.

This allows the user to view an X-ray image of the forearm in order to view longitudinal graduations 64 and inspect their position relative to the radial bone of the subject's forearm. This provides the user with a general overview of the specimen and its dimensions, and allow the user to identify the presence any foreign body or prosthesis, such as a screw, that may preclude the user from performing certain diagnoses.

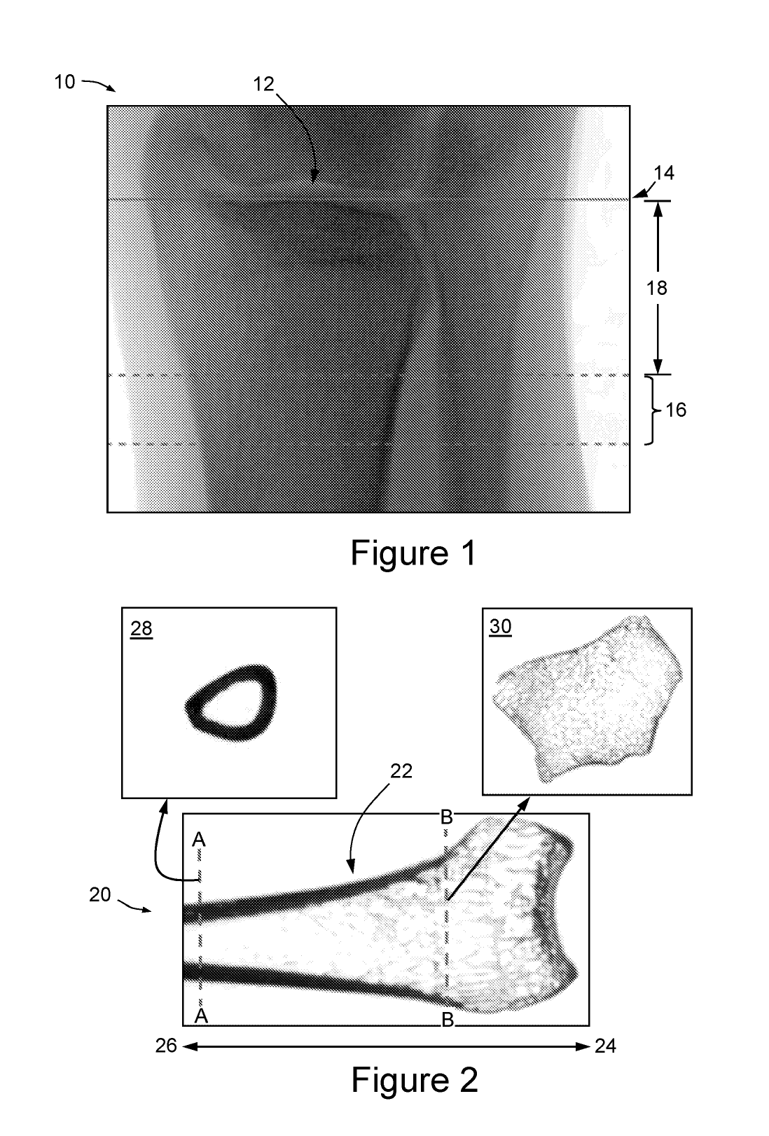

Next, system 70 will generally be controlled to obtain an overview or scout view CT scan of the forearm (cf. image 10 of FIG. 1). A scout view may be desirable if a variation of apparatus 50 is employed that lacks longitudinal graduations 64, or if the user decides to visually count longitudinal graduations 64 and so define approximately the ROI visually; in both cases the user's approximate ROI is refined subsequently by system 70.

The user then controls system 70 to scan the desired portion of the bone. As described above, the image should contain at least two circumferential graduations to allow the calculation of A′/A as image analyzer 76 processes the image. Image analyzer 76 identifies A′ and calculates the value of the relevant identifying characteristic, in this embodiment the length or width, to identify one of the two or more circumferential graduations 60 within the image.

To do so, image analyzer 76 first identifies the circumferential graduations 60 present in the image, as follows. (The following description refers to circumferential graduations 60, but a comparable approach may be applied to identify longitudinal graduations 64.) Circumferential graduations 60, as described above, are of a material of sensibly different density or densities from the other parts of apparatus 50 and are located externally to any biological tissue. As the densities differ, the attenuation, intensity, or density values in the voxels (if a CT image) or pixels (if an X-ray image) differ correspondingly.

First, graduation locater 112 of processor 90 locates circumferential graduations 60 by applying a thresholding method, optionally in combination with a prior knowledge of the position of circumferential graduations 60 in relation to apparatus 50 and hence, in general terms, relative to the rest of the forearm tissues. Graduation locater 112 may employ any suitable thresholding method, or any other segmentation method such as the method for the analysis of selected tissues disclosed in International Patent Application Publication No. WO 2011/029153.In this embodiment, graduation locater 112 uses both Otsu's method and/or that disclosed in International Patent Application Publication No. WO 2011/029153 for segmentation. These methods make any circumferential graduations 60 present in the image appear as foreground, and materials of density lower than that of circumferential graduations 60 appear as background. Graduation locater 112 then identifies connected voxels of the foreground (viz. circumferential graduations 60) that constitute a cluster of such voxels. There are two bones in the forearm, the radius and the ulna, so if only two foreground clusters are found by graduation locater 112 in the instant image slice, only bone material is present (as the radius and the ulna account for the two foreground clusters) but no circumferential graduations 60. However, in the example of the forearm, if graduation locater 112 identifies more than two clusters of foreground material in the cross-sectional slice, two foreground clusters will correspond to the radius and ulna, and the other foreground clusters will correspond to one or more circumferential graduations 60.

The output of graduation locater 112, once applied to all slices in the image of the imaged section of the radius, is used by graduation counter to count the number of circumferential graduations 60 in the imaged section. If graduation counter 114 determines that fewer than two circumferential graduations 60 are present in the image, image analyzer 76 either controls CT control system 74 to scan a longer portion of the bone (typically if image analyser 76 is connected to CT control system 74), or outputs a message to the user requesting that a longer portion of the bone be scanned (typically in the off-line mode).

Once a suitable length of the bone has been scanned, such that graduation counter 114 has determined that two or more circumferential graduations 60 appear in the image, graduation segmenter 116 of processor 90 segments the identified circumferential graduations 60. Graduation segmenter 116 may employ any suitable technique to segment each of the identified circumferential graduations 60 from the background, but in this embodiment uses a so-called ‘blob’ detection method, as follows. Firstly, graduation segmenter 116 binarizes the image using graduation locater 112 to threshold the image with several thresholds. Then, graduation segmenter 116 extracts connected components of foreground (graduation) from each binary image and calculates the centres of the identified calibration graduations 60. Next, graduation segmenter 116 groups these centres from several binary images by according to their coordinates: close centres are grouped to form a single ‘blob’, allowing segmentation of each circumferential graduation 60 in the image. As a result, the two or more circumferential graduations 60 in the image are identified and segmented on two or more CT slices, respectively.

Graduation interval determiner 118 then determines the distance between the one or more pairs of (typically consecutive) circumferential graduations 60. Graduation interval determiner 118 determines the number of slices between the pair of CT slices that contain each respective pair of identified circumferential graduation 60, and—from that result and the known slice thickness of the CT or X-ray image—determines one or more values of the distance between pairs of circumferential graduations 60 (that is, A′ when the pair of calibration graduations are consecutive, or a multiple of A′ otherwise), from which graduation interval determiner 118 determines (A′/A). It will be appreciated that in this embodiment (and others in which circumferential graduations 60 are evenly spaced), the value of A′ can be determined as the mean of a plurality of individual values, if three or more circumferential graduations 60 are identified in the image. Graduation interval determiner 118 then determines (A′/A).

In embodiments in which the spacing of the circumferential graduations 60 varies in some known manner, graduation interval determiner 118 determines one or more values of A′, and hence one or more corresponding values of (Aₙ′/Aₙ) from which graduation interval determiner 118 determines a mean value of (A′/A). Such embodiments require that the identity of the identified circumferential graduations 60 can be established, so that the correct value of Aₙ can be employed in each case, such as from the overview image or from the relationship between consecutive values of Aₙ′.

Next, graduation identifier 120 determines the characteristics of each identified circumferential graduations 60, such as length and/or width, the density of the graduation or any other predefined characteristics. In this embodiment, each calibration graduation 60 can be determined from its length.

Thus, graduation identifier 120 determines the length of a calibration graduation 60 with a contour detection algorithm that detects the contour of the calibration graduation 60 using border following. The border following of this embodiment derives a sequence of the coordinates or the chain codes from the border between a connected component of foreground and a connected component of background. The outer borders and the “hole” borders have a one-to-one correspondence to the connected components of foreground and to background, respectively, so the border following extracts the topological representation of a binary image.

In embodiments in which calibration graduation 60 can be identified from their width, graduation identifier 120 determines the width of a graduation, if required, as the number of slices in which the graduation is identifiable multiplied by the known slice thickness.

Graduation data storage 140 includes the relevant characteristic of each of circumferential graduations 60 and longitudinal graduations 64. Each of the characteristics of circumferential graduations 60 alone or in combination indicates the rank n of the respective graduation in relation to the reference point (in this embodiment, the wrist joint line). Thus, Graduation identifier 120 compares the determined length and/or width of each identified circumferential graduation 60 with the information in graduation data storage 140, and thereby identifies n for each identified circumferential graduation 60.

The results generated by graduation identifier 120 are passed to calibrator 122, which prepares a calibration of the image based on the characteristics of calibration graduations 60 determined from the image and the known values of the respective calibration graduations 60 stored in graduation data storage 140. The resulting calibration is passed to ROI selector 124 for future use. (In a variation of this embodiment, calibrator 122 is provided as a part of ROI selector 124.)

Once the calibration has been determined, image analyzer 76 then determines if an initial ROI requested by the user with ROI selector 124 is present within the image. If the ROI is not present within the image, image analyzer 76 either, in an online mode, controls CT scanner 72 to collect an image at a location that contains the requested ROI or, in an off-line mode, displays a message to the user to instruct the user as to where to collect an image so that it contains the selected ROI.

When the requested ROI is ultimately present within the image, ROI selector 124 automatically selects a region of interest based on the calibration and a predefined point or region of interest. For example, a user may wish to image a part of the radial bone 25% of the length of the bone from the wrist joint, either in the same subject but on multiple occasions, or in different subjects. ROI selector 124 can select the appropriate location for imaging each time, through the use of image ruler apparatus 50, once the location has be supplied to system 70, such as in the form of “25% of the length of the bone from the wrist joint.”

Thus, graduation locater 128, graduation counter 130, graduation segmenter 132, graduation interval determiner 134, graduation identifier 136 and calibrator 138—used in conjunction with image ruler apparatus 50—allow image analyser 76 to calibrate the image and hence reproducibly determine distances from a reference point, in this example the wrist joint, such that the locations of subsequent regions of interest can be input or output expressed as locations relative to the reference point based on that calibration. The position of a selected ROI can be expressed in relative terms (e.g. as a percent of the length of the specimen) and or in absolute terms (i.e. relative to the reference point). A selected ROI can subsequently be used for many purposes including, when—as in this example—the specimen contains bone, quantification of bone architecture and density.

The user may control system 70 to perform this calibration in several ways, including in the form of written text, identifying a region using a mouse, touchscreen or any other form of identification of selection of a given location. An output may be the location of a point touched (on a touchscreen input) by the user or a requested ROI. ROI selector 124 uses inputs from image analyser 76 to determine if a ROI initially requested by the user is present in the image and satisfactory for performing calibration. If a requested and suitable ROI is present, image analyser 76 selects and outputs an image of the requested ROI. If not, image analyser 76 requests that the user modify the image so that it has the suitable ROI and contains sufficient circumferential graduations 60.

If image analyser 76 is connected to CT control system 74 (and hence to CT scanner 72), image analyser 76 may automatically modify the field of view or other settings so that the imaged part of the specimen has a ROI containing sufficient circumferential graduations 60.

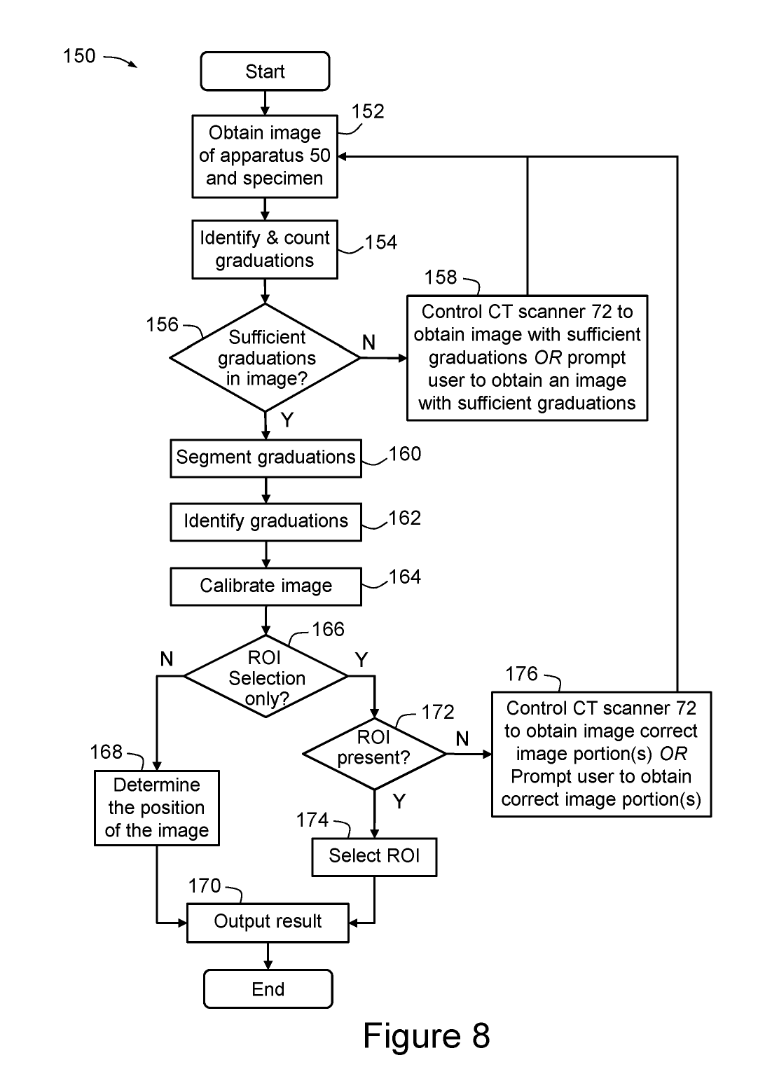

This procedure is summarized in FIG. 8, which is a flow diagram 150 illustrating the calibration process. At step 152, the forearm of the subject—wearing apparatus 50—is imaged by system 70 (or imported from another source). At step 154, image analyser 76 identifies and counts circumferential graduations 60 (using graduation locater 112 and graduation counter 114). At step 156, graduation counter 114 determines whether there are sufficient circumferential graduations 60; if not, control passes to step 158 where image analyzer 76 either controls CT scanner 72 to obtain an image with sufficient circumferential graduations 60 (such as with a longer portion of the radial bone) or sends or displays a message to the user requesting that the user obtain an image with sufficient circumferential graduations 60. Control then returns to step 152.

If, at step 156, graduation counter 114 determines that there are sufficient circumferential graduations 60 in the image, control passes to step 160 where circumferential graduations 60 are segmented by graduation segmenter 116. Next, at step 162 graduation identifier 120 identifies the circumferential graduations 60 in the image.

Next, at step 164, calibrator 122 calibrates the image based on the identified circumferential graduation and interval between graduations provided by graduation interval determiner 118.

At step 166 it is determined that the user controlled image analyzer 76 solely to determine the location of the image, processing passes to step 168 where image processor 76 applies the calibration and determines the location of the image therefrom. At step 170, the location is outputted by result output 126.

However, if at step 166 it is determined that the user controlled image analyser 76 to select a specific ROI, control passes to step 172, where ROI selector 124 determines whether the ROI requested by the user is present within the image. If ROI selector 124 determines that the selected ROI is present, control passes to step 174, where the ROI selector 124 selects the ROI. Control then passes to step 170, where an image of the selected ROI is outputted by result output 126.

If, at step 172, ROI selector 124 determines that the selected ROI is not present within the image, control passes to step 176, where either, in online operation, image analyzer 76 controls CT scanner 72 to obtain an image of the correct portion or portions of the bone or, in off-line operation, image analyzer 76 prompts the user to obtain an image of the correct portion or portions of the bone. Control then returns to step 152.

In certain embodiments, prior determination of the one or more physical characteristics of the subject or specimen being imaged is not required before the region of interest can be selected. The following descriptions are examples of how the present invention can be used to select the region of interest in such circumstances. These embodiments are envisaged to be of particular value when it is desired to collect images of an subject (such as a patient) repeatedly over an extended period of time. In such cases, it is important to collect the images at a consistent relative location, that is, with as constant a region of interest relative to the subject as possible. The absolute location may commonly be unimportant.

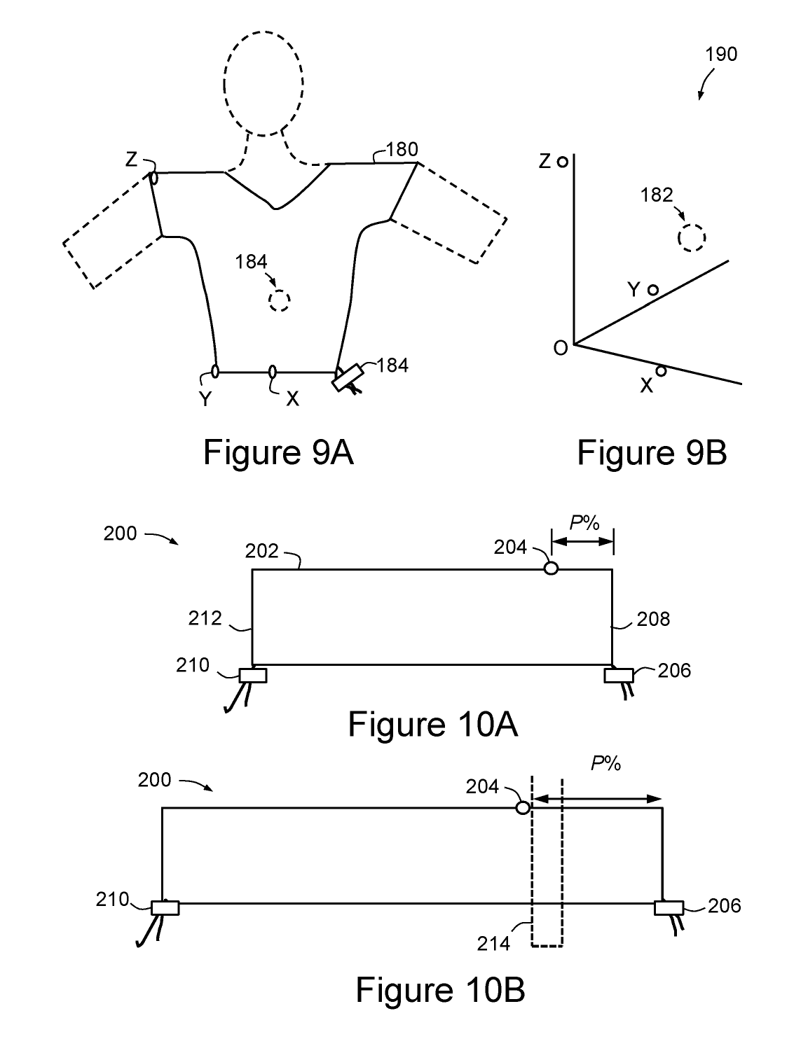

FIG. 9A is a schematic view of an image ruler apparatus 180 according to an embodiment of the present invention. In this embodiment, the selection of the region of interest (in this example, a lesion 182) does not require calibration. That is, a knowledge of the dimensions of the subject covered by apparatus 180 is not needed. In this embodiment, apparatus 180 may be designed to be wearable and may be in the form of an item of clothing, such as a shirt. Apparatus 180 may be made of a resilient material.

Apparatus 180 is provided with a fastener, which may be in the form of a cord lock 184. When worn, apparatus 180 can be fastened at the hip using fastener 184, in order to at least somewhat maintain constant measurement conditions. Apparatus 180 has embedded at predefined locations three calibration graduations X, Y and Z. Apparatus 180 is designed so that graduation X, in this example positioned anteriorly, serves as a point that allows the defining of an x-axis. Graduation Y, in this example positioned laterally, serves as a point that allows the defining of a y-axis, and graduation Z serves as a point that allows the defining of a z-axis.

In a lateral scout X-ray view, graduation X will be visible and allow the definition of the x-axis. In an antero-posterior scout X-ray view, the Z and Y graduations will be visible. A vertical line passing through graduation Z can be used to define a line parallel to the z-axis, a projection of that line to a point intersecting the x-axis defines the z-axis and the origin of the referential (O). An horizontal line passing graduation Y defines a line parallel to the y-axis. A projection of that line to a parallel passing through the referential (O) defines the actual y-axis.

This process allows identification of 3D referential system containing an origin (O) and the 3D coordinate system allows identification and selection of any region of interest and input by the user. FIG. 9B is a schematic 3D view 190 of the position of lesion 182 in relation to graduation referentials derived by calibrator 122 from calibration graduations X, Y and Z. The input may be the medio-lateral position (x-value), the antero-posterior position (y-value) or the slice number (z-value). With such inputs, the image acquisition may be limited to slices containing lesion 182, thereby reducing scanning time and radiation exposure. The 3D contours of lesion 182 can also be defined, allowing a more precise measurement of its volume.

A change in geometry of the subject being scanned—such as a patient—may influence the coordinate system. There may be a decrease in height due to a fracture or other trauma so, in such circumstances, a correction factor (e.g., the amount of decrease in height) may be applied to enable scanning of the correct region of interest. This may entail the application of a simple scaling factor. Other changes in geometry affecting the position of the graduations may similarly be identified and accounted for so that the region of interest is properly selected, so as to coincide with—in this example—lesion 182, in each subsequent scan.

FIG. 10A and 10B are schematic views of an image ruler apparatus 200 according to an embodiment of the present invention (FIG. 10A illustrating an at rest state and FIG. 10B an extended, in use state of apparatus 200). In this embodiment, the selection of the region of interest involves the selection of a fixed percentage of the length of the subject being imaged and thus does not require an absolute calibration. Indeed, it is not necessary to establish the actual length of the subject (patient or otherwise) being scanned.

In this embodiment, apparatus 200 includes an elongate, resilient member 202 with an identifiable graduation 204 located at a predetermined percentage P % of the length of member 202, corresponding to the percentage along the subject at which an image is to be collected. Member 202 is made so that the extension or compression is uniform along its length. Under these circumstances, the relative position of graduation 204 does not change. Apparatus 200 also includes a fastener for fastening member 202 to the subject in the at rest and in use forms, which may be in the form of a first tie 206 located at a proximal end 208 of member 202 (such as the wrist end when adapted for use on a forearm) and a second tie 210 located at a distal end 212 of member 202 (such as the elbow end when adapted for use on a forearm).

Referring to FIG. 10B, CT control system 74 may control CT scanner 72 to collect images encompassing graduation 204 or with graduation 204 marking the proximal or distal end of the image, according to the desired region of interest 214. In the illustrated example, graduation 204 is used to mark the distal end of region of interest 214. It is should be noted that on this embodiment, one graduation is sufficient, though more may be employed.

Thus, in this embodiment the position of graduation 204 relative to a preset referent value is known, so CT scanner 72 can be controlled to select the correct region of interest 214 based on visual identification within the image of graduation 204 and the known position of graduation 204 relative to the dimensions of the subject being imaged.

Once the correct region of interest 214 is identified either automatically or manually, region of interest 214 may be outputted.

In certain embodiments, and in particular that described by reference to FIGS. 10A and 10B, it will be evident that the graduation may be straightforwardly made adjustable in position. For example, graduation 204 may be mounted slidably on a cord attached to proximal end 208 and distal end 212 of member 202. The cord may be of a material, such as nylon, that resists inadvertent displacement of graduation 204, and be marked with successive position markers corresponding to, for example, P=5%, 10%, 15%, 20%, etc. This would allow the user to set the position of graduation 204 as desired before use.

Many other position adjustment mechanisms are possible, such as snap fasteners (i.e. press studs, poppers, snaps or tiches) placed at regular percentage intervals along member 202, with a movable complementary portion comprising the graduation that and can be moved by the user to the desire percentage of the length of member 202. Alternatively, the position adjustment mechanism may comprise a strip or pieces of velcro and a movable complementary portion comprising the graduation, or a zipper with the movable locking mechanism provided with the graduation.

Modifications within the scope of the invention may be readily effected by those skilled in the art. For example:
(i) variations of apparatus 50, 180 or 200 are of different dimensions as appropriate for use with other human bones or with non-human bones, or other human or non-human body part;
(ii) when graduations are made of materials of different densities, in addition to the anatomical calibration described above, these differences in density can be used by image analyzer 76 to perform a density calibration of the bone present within the image using a conventional bone density calibration protocol;
(iii) in some embodiments of the invention, an image ruler apparatus is provided that, rather than being extensible, is compressible between a relaxed form and a compressed form; in the relaxed form, such an image ruler apparatus may be longer and/or of greater perimeter than the limb (or other part of the body) with which it is to be used, compressed to the required extent in situ, then fastened in place; it will be appreciated that the above description, suitably modified, also serves to explain the operation of such alternative embodiments.

It is to be understood, therefore, that this invention is not limited to the particular embodiments described by way of example hereinabove.

In the claims that follow and in the preceding description of the invention, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments of the invention.

Further, any reference herein to prior art is not intended to imply that such prior art forms or formed a part of the common general knowledge in any country.

1. An apparatus for calibrating an image of a specimen or subject obtained with an imaging modality or selecting a region of interest in the image, the apparatus comprising:
a graduation support adapted for mounting on the specimen or subject; and
one or more calibration graduations supported by the graduation support;
wherein the one or more calibration graduations are imagable with the imaging modality, and are distinguishable in the image from the graduation support and from the specimen or subject; and
wherein at least one of the calibration graduations have at least one characteristic of known value.

2. An apparatus as claimed in claim 1, wherein:
the graduation support comprises an elongate member extensible or compressible between a relaxed form and an extended or compressed form; and
the one or more calibration graduations are provided along the elongate member;
wherein the one or more calibration calibration graduations are imagable with the imaging modality, and are distinguishable in the image from the elongate member and from the specimen or subject; and
wherein at least some of the calibration graduations have at least one characteristic of known value in the relaxed form and of determinable value from the image.

3. An apparatus as claimed in claim 1, wherein the graduation support:
i) is resilient;
ii) comprises an elastic material, one or more springs or a telescopic mechanism;
iii) is extensible between the relaxed form and an extended form and compressible between the relaxed form and a compressed form; and/or
iv) comprises an elongate member.

4-5. (canceled)

6. An apparatus as claimed in claim 1, wherein all of the calibration graduations have at least one characteristic of known value and of determinable value from the image.

7. An apparatus as claimed in claim 1, wherein an identity of at least one of the calibration graduations is determinable from the image.

8. An apparatus as claimed in claim 1, wherein the at least one characteristic is (i) a known spatial position of one or more of the graduations, (ii) a separation of each of the calibration graduations from one or more immediately neighbouring calibrations, (iii) the number of graduations at predetermined spatial positions, or (iv) a length of at least some of the calibration graduations.

9. (canceled)

10. An apparatus as claimed in claim 1, wherein the apparatus is generally tubular and:
i) the calibration graduations are arranged circumferentially; or
ii) some of the calibration graduations are arranged circumferentially and some of the calibration graduations are arranged longitudinally.

11. (canceled)

12. An apparatus as claimed in claim 1, wherein the calibration graduations are arranged longitudinally.

13. (canceled)

14. An apparatus as claimed in claim 1, wherein the calibration graduations:
i) have at least two different densities;
ii) have different densities from each other;
iii) a different density or densities from a density of the graduation support and a density of the specimen or subject in order to be distinguishable in the image from the graduation support and from the specimen or subject; and/or
iv) are located at respective predetermined spatial positions so as to be distinguishable in the image from the graduation support and from the specimen or subject;
v) are located at respective predetermined spatial positions so that the spatial positions can be used as respective referential points.

15-17. (canceled)

18. An apparatus as claimed in claim 1, comprising a fastener for fastening the graduation support to a specimen or subject in the extended or compressed form.

19-20. (canceled)

21. A method for calibrating an image of a specimen or subject obtained with an imaging modality or selecting a region of interest in the image, the method comprising:
locating an apparatus as claimed in I on a subject or specimen;
imaging at least a portion of the subject or specimen containing at least one of the calibration graduations;
determining from the image the at least one characteristic of one or more of the calibration graduations; and
selecting the region of interest.

22. A method as claimed in claim 21, including comparing the at least one characteristic of the one or more of the calibration graduations in the image with known values of the respective one or more calibration graduations in the image and determining a calibration therefrom.

23. (canceled)

24. A method as claimed in claim 21, including determining from the image the at least one characteristic of at least two of the calibration graduations, and/or automatically selecting or identifying a region of interest for imaging based on the calibration and a predefined or desired imaging location.

25. A method as claimed in claim 21, wherein locating the calibration apparatus on the subject or specimen includes extending or compressing the graduation support. (Original)

26. A method as claimed in claim 21, wherein comparing the at least one characteristic of the one or more of the calibration graduations in the image with known values of the respective one or more calibration graduations in the image comprises determining a ratio of relaxed and extended or compressed separations of one or more pairs of calibration graduations, or determining a ratio of relaxed and extended or compressed lengths or widths of one or more calibration graduations, or counting the number of graduations arranged circumferentially, longitudinally, or any other direction.

27. A method as claimed in claim 21, wherein an identity of at least one of the calibration graduations is determinable from the image.

28. A method as claimed in claim 21, wherein the at least one characteristic is:
i) a separation of each of the calibration graduations from one or more immediately neighbouring calibrations;
ii) a length of at least some of the calibration graduations; and/or
iii) the number of calibration graduations in a given direction.

29. (canceled)

30. A method as claimed in claim 21, wherein the graduation is unique and identifiable by at least one of its characteristics.

31. (canceled)

32. A method as claimed in claim 21, wherein the calibration graduations have at least two different known densities, and the method includes calibrating a specimen or subject density based on the at least two different known densities of the calibration graduations.

33. A system for calibrating an image of a specimen or subject obtained with an imaging modality or selecting a region of interest in the image, the system comprising:
an input for receiving an image of the specimen or subject and of an apparatus as claimed in claim 1;
a calibration graduation locater for locating one or more calibration graduations in the image;
a graduation segmenter for segmenting the calibration graduations in the image;
a graduation identifier for identifying at least one characteristic of the calibration graduations in the image; and
a calibrator for preparing a calibration of the image based on the at least one characteristic of the calibration graduations in the image.

34. A system as claimed in claim 33, wherein the calibrator prepares the calibration based on the at least one characteristic of the calibration graduations in the image and known values of the respective one or more calibration graduations in the image.

35. A system as claimed in claim 33, wherein the calibration system is configured to output the calibration, and/or to use the calibration in subsequent image analysis.

36. A system as claimed in claim 33, wherein the one or more calibration graduations have at least two different known densities, and the calibrator is configured to calibrate a specimen or subject density based on the at least two different known densities of the calibration graduations.

37. An image analysis system or an imaging system, comprising a system as claimed in claim 33.

38. (canceled)

39. A non-transitory computer-readable medium comprising a computer software product configured to control one or more processors to implement a method as claimed in claim 21.

40. (canceled)
